(19) United States
(12) Patent Application Publication
(10) Pub. No.: US 2023/0000328 A1
(43) Pub. Date: Jan. 5, 2023

(54) ENDOSCOPE DEVICES COMPRISING A MOVEABLE CAMERA

(71) Applicant: MEDIGUS LTD., Omer (IL)

(72) Inventor: Amir GOVRIN, Ramat Gan (IL)

(21) Appl. No.: 17/930,495

(22) Filed: Sep. 8, 2022

Related U.S. Application Data
(63) Continuation-in-part of application No. PCT/IL2021/050245, filed on Mar. 7, 2021.
(60) Provisional application No. 62/988,466, filed on Mar. 12, 2020.

(51) Int. Cl. A61B 1/05; A61B 17/32; A61B 1/06

(52) U.S. Cl. CPC A61B 1/053; A61B 17/32002; A61B 1/06; A61B 2017/003

(57) ABSTRACT

Disclosed is an endoscopic device comprising an insertion tube, a distal tip, and at least one camera located at an initial location on or in the distal tip or the insertion tube. The endoscopic device is characterized in that it comprises a mechanical arrangement that is activated from the proximal end of the endoscopic device and is configured to do at least one of the following: (a) to move the at least one camera from the initial location to one or more different locations on, in or spaced away from the distal tip or the insertion tube; and (b) to change the direction of view of the at least one camera. Also disclosed are methods of performing endoscopic procedures using embodiments of the endoscopic device.

### FIELD OF THE INVENTION

The present invention relates to the field of endoscopic devices. Specifically, the invention relates to visualization means provided on endoscopic devices.

### BACKGROUND OF THE INVENTION

Endoscopic devices are devices comprising a handle (control section), an insertion tube (insertion shaft), typically visualization and illumination means on the distal face, and means for transmitting images captured by the visualization means to a display device. The insertion shaft can be either rigid, flexible or a combination of both. It can also comprise an articulation section.

Endoscopic devices that are used in medical procedures are known inter alia as endoscopes or laparoscopes. They are inserted into a patient's body either through a small cut or through a natural opening in the body such as the mouth, in order to inspect internal organs. Endoscopes are used to perform surgical procedures, such as removal of abnormal tissues, e.g. tumors or polyps by means of additional devices that are either mounted on the insertion shaft or introduced to the surgical location via one or more working channels through the insertion shaft. Endoscopic devices, e.g. borescopes, are used in industrial applications. Herein the word endoscope is used to refer to all types of endoscopic devices both medical and industrial.

Many different types of visualization means e.g. CCD or CMOS still or video cameras, optical relay systems, optical fibers, “chip on a stick”, etc., are known in the art of endoscopy for transferring images proximally from the distal to the proximal end of the insertion tube, Since most modern endoscopic devices comprise video cameras, herein (including the claims) the word camera is used to represent any of these systems.

All endoscopic devices known to the inventors comprise one or more cameras that are fixedly attached to either the distal tip or the insertion shaft of the endoscope. These cameras have a direction of view and field of view that is predetermined according to the intended use of the endoscope. In certain situations this field of view or direction of view is inadequate to carry out the intended task and in other situations the field of view becomes partially or totally blocked, for instance during a medical procedure when tissue that is being operated upon comes between the lens of the camera and the surgical tool. In these situations the surgeon either has to carry out the procedure “blind” or to reposition the endoscopic device in an attempt to get a better view. It would therefore be advantageous to have an endoscopic device capable of obtaining images from a variety of fields of view and of maintaining continuous endoscopic observation throughout an entire endoscopic procedure without the necessity of rotating or repositioning the endoscope.

It is therefore an object of the present invention to provide an endoscopic device capable of providing images from multiple directions of view and corresponding fields of view during an endoscopic procedure.

It is another object of the present invention to provide an endoscopic device capable of maintaining continuous endoscopic observation throughout an entire endoscopic procedure.

Further purposes and advantages of this invention will appear as the description proceeds.

### SUMMARY OF THE INVENTION

In a first aspect the invention is an endoscopic device comprising an insertion tube, a distal tip, and at least one camera located at an initial location on or in the distal tip or the insertion tube. The endoscopic device is characterized in that it comprises a mechanical arrangement that is activated from the proximal end of the endoscopic device and is configured to do at least one of the following:
a) to move the at least one camera from the initial location to one or more different locations on, in or spaced away from the distal tip or the insertion tube; and
b) to change the direction of view of the at least one camera.

Embodiments of the endoscopic device comprise a first set of cameras comprising one or more cameras that are fixedly attached to the distal tip or insertion tube of the endoscopic device and a second set comprising one or more cameras each of which is mounted on at least one rod configured to be reversibly extended outwards from the distal tip or insertion tube of the endoscopic device. The first set of cameras provides a direction of view looking outwards from the interior of the endoscopic device and the second set of cameras provides a direction of view looking inwards towards the endoscopic device.

In embodiments of the endoscopic device comprising a first and a second set of cameras the at least one rod can be configured to be reversibly extended in any direction with respect to the surface of the endoscope in order to provide a field of view that cannot be obtained by cameras that are attached fixedly to the endoscope.

In embodiments of the endoscopic device at least one of the cameras is mounted in such a way that the camera can be panned, tilted, or rotated in order to provide various angles of view without rotating or moving the endoscope.

In embodiments of the endoscopic device comprising a first and a second set of cameras the first set of cameras comprises one front facing camera fixedly attached to distal face of the distal tip and the second set of cameras comprises one backward facing camera mounted on a camera mount attached to at least one rod that can be extended from a first position abutting a base to a second position spaced apart from the base and retracted proximally from the second position back to the first position. The base can be one of:
a) a specially configured distal tip, which is an integral part of a dedicated endoscope, wherein the distal tip comprises at least one bore through which the at least one rod can be slid distally and proximally; and
b) a collar which slips over and is attached to the distal tip of an endoscope, wherein the collar comprises at least one bore through which the at least one rod can be slid distally and proximally.

This embodiment of the endoscopic device can comprise at least one of: (i) a stapling device comprised of an anvil and a staple cartridge, wherein the anvil is mounted on two rods which can be extended distally to obtain an opened configuration or can be retracted proximally to obtain a closed configuration; and wherein the staple cartridge comprises a distal section configured to slide proximally into a proximal section of the cartridge; (ii) at least one tissue gripping device comprising at least one tissue gripper configured to be reversibly extended from the base; and (iii) at least one cutting device configured to cut through tissue.

In embodiments of the endoscopic device comprising a stapling device wherein the anvil is attached to two rods that have a straight configuration when in a first location and a bowed configuration when in a second location.

In embodiments of the endoscopic device the mechanical arrangement is a ball joint located on a distal face of the distal tip. The ball joint comprises a ball on which at least one camera and illumination sources are mounted. The ball is configured to be rotated by an operator of the endoscopic device by activating cables or an electric motor and gear system, thereby changing the direction of view of the at least one camera.

In embodiments of the endoscopic device the mechanical arrangement is a rotatable collar on which is mounted the at least one camera and illumination sources. The collar is configured to be rotated by an operator in a clockwise or counterclockwise direction around a longitudinal symmetry axis of the insertion tube by rotating a cable connected to the collar or by activating an electric motor and gear system, thereby changing the direction of view of the at least one camera.

These embodiments of the endoscopic device may comprise at least one additional camera on the distal tip. The at least one additional camera has one of:
a) a fixed front facing direction of view; and
b) a mechanical arrangement configured to change its direction of view.

In a second aspect the invention is a method of performing an endoscopic closure procedure, said method comprising the steps:
a) inserting an endoscopic device comprising a front facing camera, a backward facing camera, a stapling device comprised of an anvil and a staple cartridge, wherein the anvil is mounted on two rods which can be extended distally to obtain an opened configuration or can be retracted proximally to obtain a closed configuration; and wherein the staple cartridge comprises a distal section configured to slide proximally into a proximal section of the cartridge and at least one tissue gripping device into the body of a patient and using the front facing camera to guide the insertion tube to the location of a hole, which is to be closed in tissue of the patient;
b) using the front facing camera to position the distal tip of the endoscopic device above and parallel to the hole which is to be closed;
c) actuating wires which cause the anvil of the stapling device and the camera mount to move distally;
d) actuating the at least one tissue gripper to extend and firmly grasp tissue around the hole;
e) pulling the tissue gripper and attached tissue upwards and proximally until all tissue surrounding the hole that is to be closed is above slots through which staples in stored in the proximal section of the staple cartridge are ejected from the distal section of the cartridge;
f) using the backward facing camera to ensure the tissue is correctly positioned relative to the staple cartridge;
g) retracting the anvil towards the staple cartridge thereby pressing the tissue surrounding the hole between the anvil and the cartridge;
h) continuing to retract the anvil towards the staple cartridge, thereby ejecting an array of staples from the staple cartridge through the tissue;
i) moving the anvil distally thereby releasing the stapled tissue and using the backward facing camera to inspect and verify that the stapling procedure has been properly executed; and
j) withdrawing the endoscope from the body of the patient.

In a third aspect the invention is a method of performing a Full Thickness Resection (FTR) procedure to remove a growth, the method comprising the steps:
a) Inserting an endoscope comprising a front facing camera, a backward facing camera, a stapling device comprised of an anvil and a staple cartridge, wherein the anvil is mounted on two rods which can be extended distally to obtain an opened configuration or can be retracted proximally to obtain a closed configuration; and wherein the staple cartridge comprises a distal section configured to slide proximally into a proximal section of the cartridge, at least one tissue gripping device, and a cutting device into the body of a patient and using the front facing camera to guide the endoscope to the location of a growth that is to be removed;
b) using the front facing camera to position the endoscope above and parallel to the growth;
c) actuating wires which cause the anvil and the camera mount to move distally;
d) adjusting the position of the endoscope using both the front facing camera and the rear facing camera so that the growth is correctly positioned below and in front of the distal tip of the endoscope;
e) actuating the tissue gripper to firmly grip the growth;
f) retracting the gripper, thereby pulling the entire growth above the staple cartridge until a portion of tissue surrounding the growth is located opposite slots in the distal section of the cartridge through which staples in the proximal section of the cartridge are ejected from the distal section of the cartridge;
g) using the backward facing camera to ensure that the growth is in the correct position;
h) retracting the anvil towards the staple cartridge thereby pressing tissue surrounding the growth between the anvil and the staple cartridge;
i) continuing to retract the anvil towards the the staple cartridge, thereby ejecting at least one staple from the staple cartridge through the tissue surrounding the growth;
j) actuating the cutting device thereby cutting the tissue surrounding the growth below the staple line;
k) moving the anvil distally thereby releasing the stapled tissue while leaving the growth connected to the gripper; and
l) withdrawing the growth together with the endoscope from the body of the patient.

An embodiment of the third aspect of the invention comprises an additional step introduced between steps i and j. The additional step comprises moving the anvil distally after firing the staples to carry out a detailed inspection of the stapled tissue using the backward facing camera followed by moving the anvil proximally before cutting the tissue.

In a fourth aspect the invention is a robotic surgical system comprising at least one endoscopic device of the first aspect.

All the above and other characteristics and advantages of the invention will be further understood through the following illustrative and non-limitative description of embodiments thereof, with reference to the appended drawings.

### BRIEF DESCRIPTION OF THE DRAWINGS

FIG. 1 schematically shows a front view of an endoscope comprising visualization means according to the present invention;

FIGS. 2A and 2B are schematic perspective views of the distal end of the endoscope of FIG. 1 in an open configuration;

FIG. 3 schematically shows a side view of the endoscopic device of FIG. 1 in its opened configuration;

FIG. 4 schematically shows a bottom view of the endoscopic device of FIG. 1 in its opened configuration;

FIG. 5 schematically shows an add-on embodiment of the endoscopic device of FIG. 1 in its opened configuration surrounded by a sheath;

FIG. 6A is a flowchart describing an endoscopic closure process using the endoscope of FIG. 1;

FIG. 6B is a flowchart describing a full thickness resection process using the endoscope of FIG. 1;

FIG. 7 schematically shows a perspective view of an anvil mounted on rods made of shape memory material;

FIGS. 8A to 8F schematically show different views of the distal tip of an endoscopic device comprising a camera and illumination sources mounted on the ball of a ball joint;

FIGS. 9A to 9C schematically show different views of the distal tip of an endoscopic device comprising a rotatable collar on which is mounted a camera and illumination sources; and

FIGS. 10A to 12C schematically show different views of embodiments of the distal tip of an endoscopic device that comprises a camera mounted on a camera mount whose location can be moved from an initial position in a socket in the distal tip to a location separated by a distance from the distal tip.

### DETAILED DESCRIPTION OF EMBODIMENTS OF THE INVENTION

The present invention is an endoscopic device comprising an insertion tube (for a flexible device or insertion shaft for a rigid device—henceforth herein the term “insertion tube” will be used for both cases), a distal tip, and at least one camera located at an initial location on or in the distal tip or the insertion tube. The endoscopic device also comprises a mechanical arrangement that is activated from the proximal end of the endoscopic device. The mechanical arrangement is configured to do at least one of the following: (a) to move the camera from the initial location to one or more different locations on, in or spaced away from the distal end of the insertion tube; and (b) to change the direction of view of the camera, i.e. the direction in which the optical axis of the camera is pointed relative to the distal direction defined by the longitudinal symmetry axis of the insertion tube.

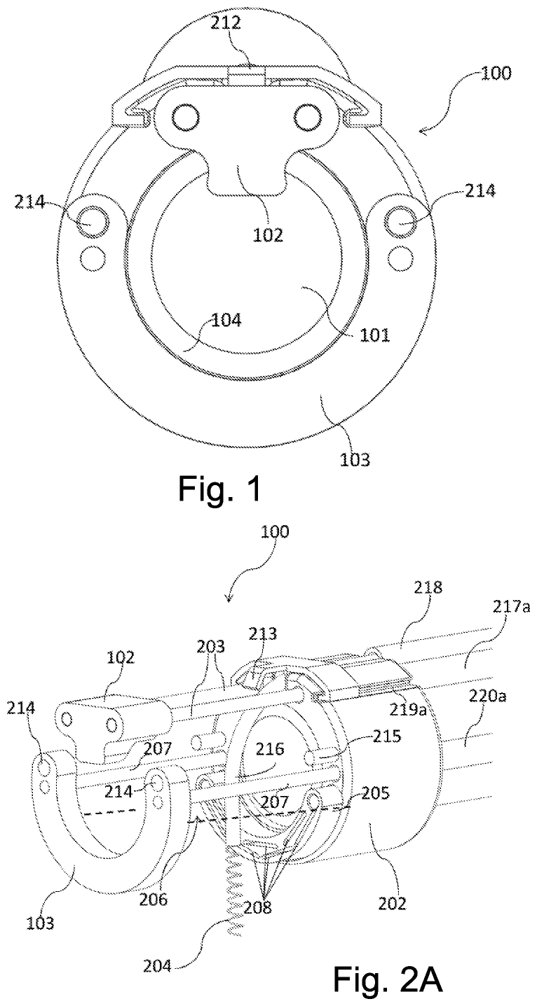

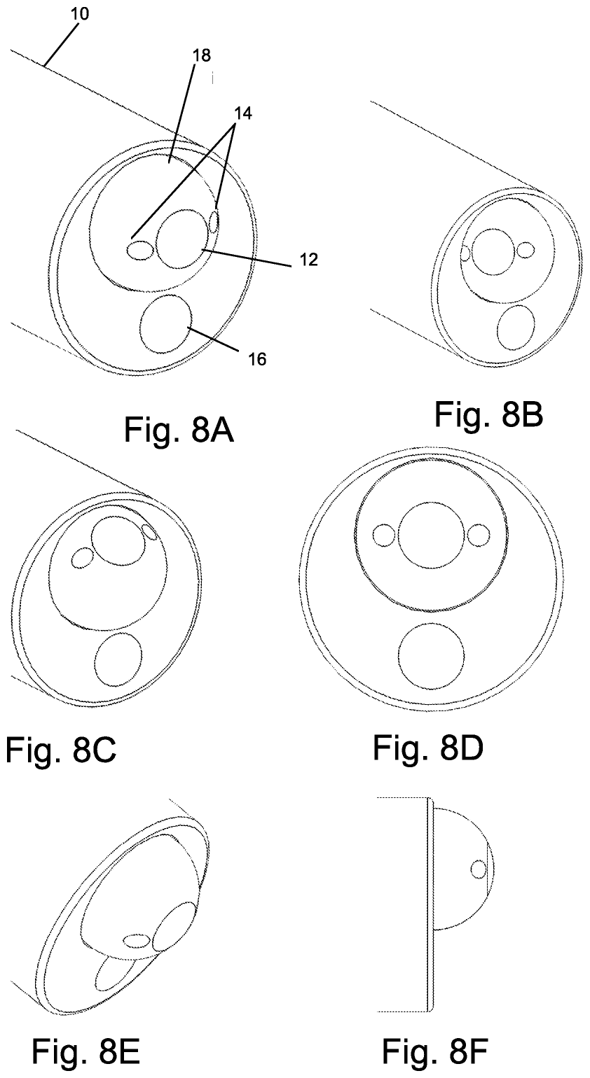

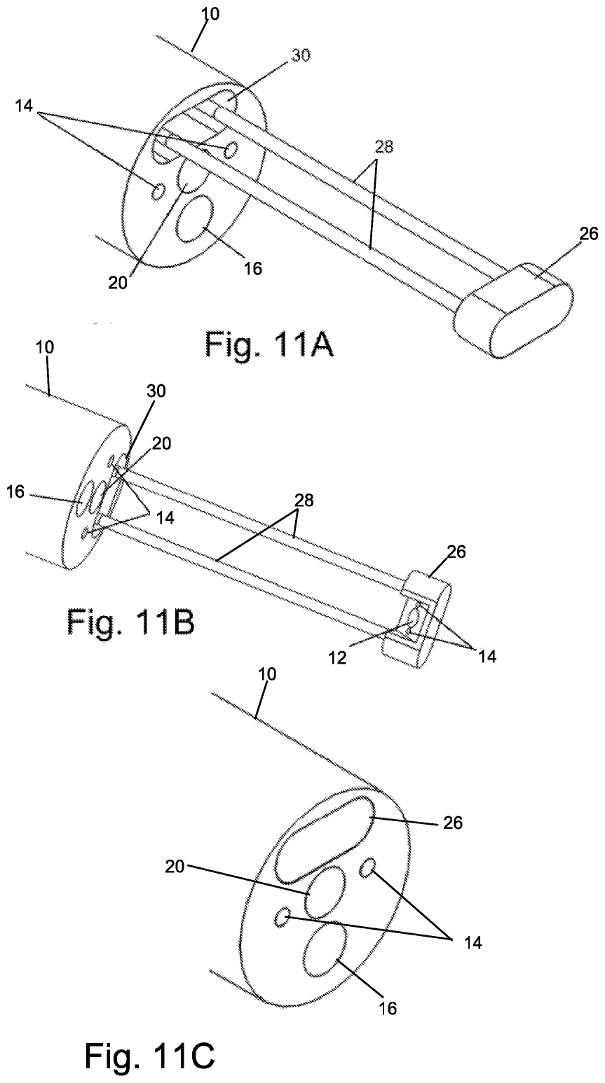

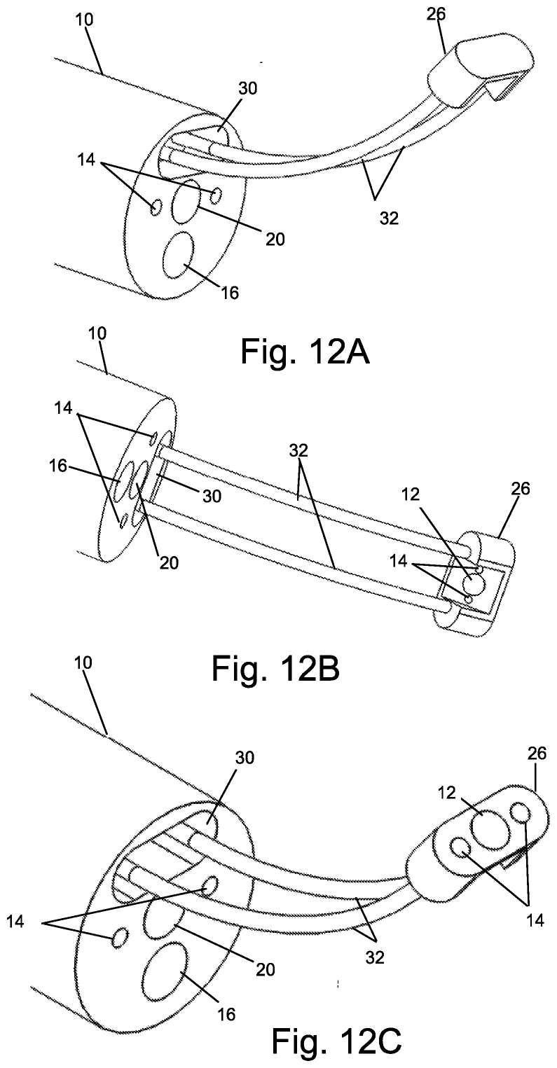

The invention can be implemented in many different ways. FIGS. 8A to 12C schematically illustrate some of these ways. FIGS. 1 to 7 schematically illustrate in more detail the embodiment shown in FIGS. 11A to 12B.

FIGS. 8A to 8F schematically show different views of the distal tip 10 of an endoscopic device. In this embodiment the mechanical arrangement is a ball joint comprising a ball 18 on which a camera 12 and illumination sources 14, e.g. LEDs or the distal ends of optical fibers, are mounted. Also seen in the figures is the distal end of a working channel 16. The ball 18 can be rotated by an operator of the endoscopic device by activating cables or a small electric motor and gear system thereby changing the direction of view of the camera as seen in—these figures.

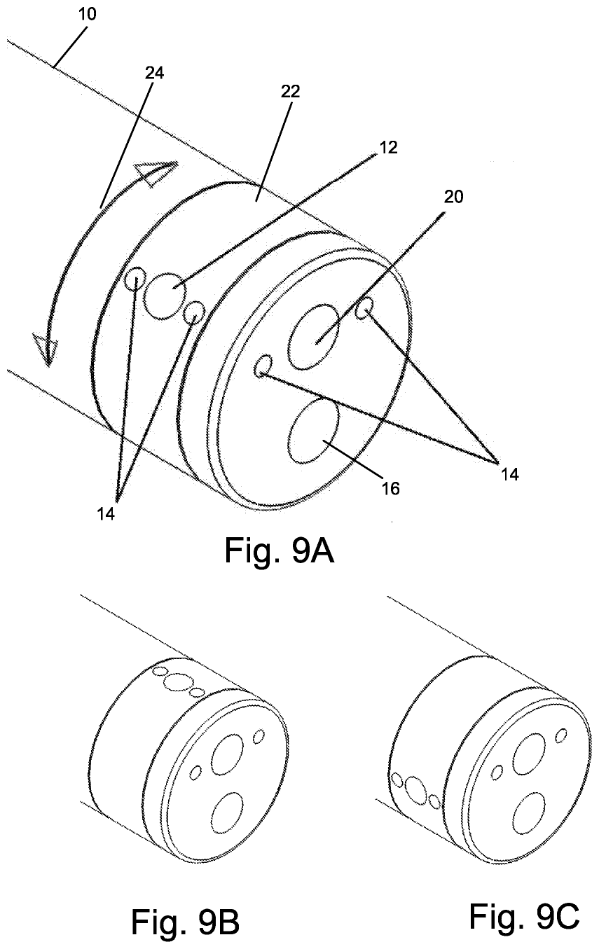

FIGS. 9A to 9C schematically show different views of the distal tip 10 of an endoscopic device comprising a rotatable collar 22 on which is mounted a camera 12 and illumination sources 14. Also seen in the figures are a second camera 20 and illumination sources 14 on the distal face of the distal tip. Camera 20 can have a fixed forward looking direction of view or its direction of view can be changed by a mechanical arrangement. An operator of the endoscopic device can move collar 22 in the direction indicated by double headed arrow 24 by rotating a cable connected to the collar or by activating a small electric motor and gear system thereby changing the direction of view of the camera as seen in the figures.

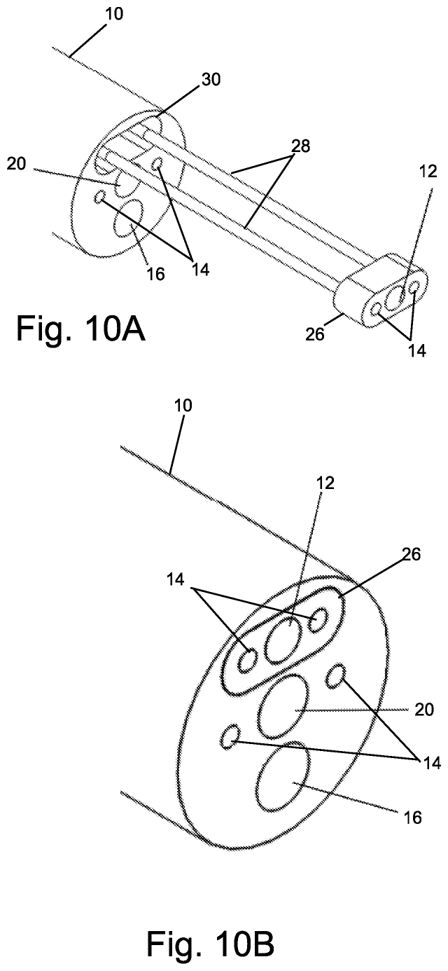

FIGS. 10A to 12C schematically show different views of embodiments of the distal tip 10 of an endoscopic device that comprises a camera 12 and illumination sources 14 mounted on a camera mount 26 whose location can be moved from an initial position in a socket 30 in the distal tip to a location separated by a distance from the distal tip. In all of these figures also seen on the distal tip are a 2′ camera 20, illumination sources 14, and a working channel 16.

FIG. 10A shows camera mount 26 supported by rods 28 that have been activated by a mechanism described in detail herein below to push camera mount 26 out of socket 30 into a location in front of distal tip 10. In this embodiment camera 14 is mounted on the front side of camera mount 26 giving it a forward looking direction of view. FIG. 10B shows the embodiment of FIG. 10A with the rods 28 pulled distally pulling camera mount 26 into socket 30.

FIGS. 11A to 11C schematically show an embodiment that is essentially the same as that shown in FIGS. 10A and 10B with the exception that camera 12 is mounted on the bottom of camera mount 26 (see FIG. 11B). In this embodiment the fields of view of cameras 12 and 20 overlap each other in front of the distal tip. This allows an object located in front of the distal tip to be imaged from different directions of view.

In FIGS. 12A to 12C, the rods 32 are not straight as are rods 28 in the previous figures. Rods 32 can be made from a shape memory material or can comprise an articulation section. This allows the camera 12 to be distanced from the distal tip and also to have a direction of view that is off-axis as compared to the longitudinal axis of the insertion tube of the endoscopic device.

The embodiments of the invention described herein are for the sake of illustration only. Many other ways of implementing the invention are possible. For example, the endoscopic device may comprise at least one camera attached fixedly to the distal tip or insertion tube of the endoscope and at least one camera attached to the distal tip or insertion tube of the endoscope by one or more rods configured to be reversibly extended in any direction with respect to the surface of the endoscopic device in order to provide a field of view that cannot be obtained by the cameras attached fixedly to the endoscope. In another example, one or more of the cameras, for instance but not necessarily, a fixed camera on the distal tip or one of the cameras attached to the endoscope by an extendible rod or rods, may be mounted in such a way that the camera can be panned, tilted, or rotated in order to provide various directions of view. In other examples different permutations of the components described herein can be utilized, for example, the moveable camera 12 or the fixed 2^{nd} camera 20 in FIGS. 10A to 12C could be replaced with a camera mounted on a ball joint as in FIGS. 8A to 8F.

Embodiments of the present invention are endoscopic devices comprising visualization means comprised of a first set of cameras comprising one or more cameras that are fixedly attached to the distal tip or insertion tube of the endoscopic device and a second set of cameras each of which is mounted on one or more rods configured to be reversibly extended outwards from the distal tip or insertion tube of the endoscopic device, wherein the first set of cameras provides a field of view looking outwards from the interior of the endoscopic device and the second set of cameras provides a field of view looking inwards towards the endoscopic device.

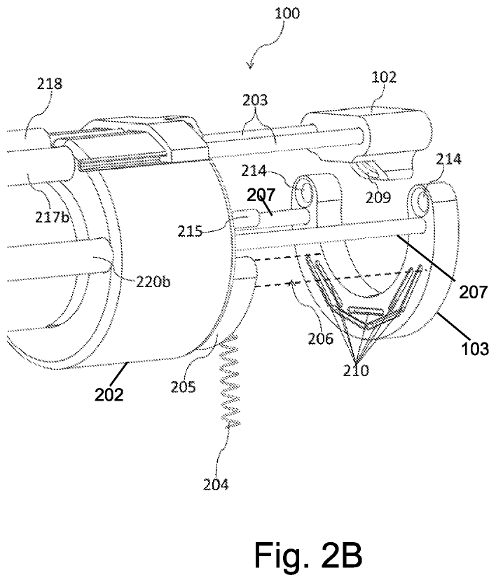

FIG. 1 schematically shows a front view of an embodiment of an endoscope 100 that comprises visualization means according to the present invention. FIG. 1 illustrates the location of a forward facing camera 101, in the center of the distal face of endoscope 100, and an illumination source, which in this embodiment is a light ring 104. In other embodiments the illumination source can be of any other type known in the art, e.g. LEDs or optical fibers. The endoscope may also comprise other conventional components such as working channels and a channel for irrigation or insufflation, which are not shown on the distal tip to simplify the figures. A camera mount 102 is located at the top of the distal tip. Camera mount 102 comprises a backward facing camera (shown in FIG. 2B) and an illumination source (not shown in the figures). At the bottom side of the distal tip 100 is seen a stapler anvil 103.

FIGS. 2A and 2B are schematic perspective views of the distal end of endoscope 100 in an open configuration. Seen in FIG. 2A and FIG. 2B are backward facing second camera (209 in FIG. 2B), which is mounted on camera mount 102 together with an illumination source, which is not shown in the figures. In this embodiment camera mount 102 is attached to two rods 203 that can be extended distally from a first position abutting base 202 to a second position spaced apart from base 202 and retracted proximally from the second position back to the first position.

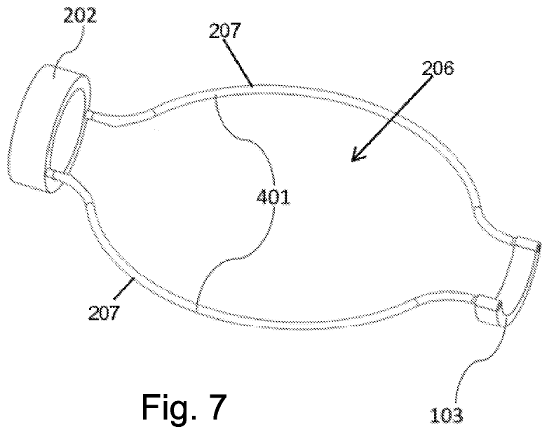

In one embodiment of the invention, the base 202 is a specially configured distal tip, which is an integral part of a dedicated endoscope. In this embodiment the distal tip comprises two bores through which rods 203 can be slid distally and proximally. In another embodiment of the invention, base 202 is a collar which slips over and is attached to the distal tip of a standard endoscope. In this embodiment the collar comprises two bores through which rods 203 can be slid distally and proximally. In this add-on embodiment, an elastomeric sheath (see FIG. 7) is used to cover operation wires for moving the rods and electrical wires to the camera and illumination means on camera mount 102 and signal wires that are located outside of the insertion tube of the endoscope, as will be described herein below.

Extendable camera rods 203 are configured to move camera mount 102 distally away from and proximally towards base 202. The motion of camera rods 203 is controlled by cables, rods or other mechanical means (not shown) that are attached to the proximal ends of rods 203. For simplicity, any arrangement known in the art for moving the rods laterally is referred to hereinafter as wires. The wires are stiff enough to cause camera rods 203 to move distally when the wires are pushed distally. In the embodiments in which base 202 is an integral part of an endoscope, the wires pass through channels in the insertion tube of the endoscope to a handle where the proximal end of the wires are attached to a mechanism that is configured to extend and retract camera rods 203. In the embodiments in which base 202 is added on to an endoscope, the wires pass through tubes 217a and 217b that are held against the insertion shaft of the endoscope by a sheath, to a dedicated control box located at the proximal end of the endoscope.

A stapling device comprised of an anvil 103 and a staple cartridge 205 is located on base 202. The designs of cartridge 205 and anvil 103 have been previously described in WO2007/141776 to the applicant of the present patent application. Anvil 103 is mounted on extendable rods 207 which can be extended distally to obtain an opened configuration (shown in FIG. 2A), or can be retracted proximally to obtain a closed configuration (shown in FIG. 1). The motion of rods 207 is controlled by a mechanical, pneumatic, or hydraulic arrangement similar to that described herein above for rods 203. In embodiments in which base 202 is an integral part of the endoscope, the wires that control movement of rods 207 pass through channels in the insertion tube of the endoscope to a handle where the proximal end of the wires is attached to a mechanism that is configured to extend and retract rods 207. In embodiments in which base 202 is added to the endoscope, the wires pass through tubes 220a and 220b that are enclosed by the additional sheath. When anvil 103 is retracted, i.e. when rods 207 are moved proximally, alignment pins 215, which are fixedly attached to base 202, enter alignment openings 214 in anvil 103 causing the anvil to be perfectly aligned with cartridge 205. In other embodiments, arrangements such as an ultrasound or optical sensor system can be used instead of the alignment pins 215 and openings 214 to obtain and verify alignment of the anvil and cartridge.

In this design of the stapler, stapler cartridge 205 does not comprise an arrangement of cams to actively eject the staples, such as is conventionally found in medical staplers. In the present stapling device, when anvil 103 is retracted proximally as described herein above, the face of anvil 103 engages a portion of tissue which is between the anvil and cartridge 300, and presses the portion of tissue against the distal face of cartridge 300, whereupon continued retraction of anvil 103 causes a distal section of cartridge 205 to slide proximally into a proximal section. Neither staple pushers nor staples move inside the proximal section and the legs of the staples are passively forced to exit the distal section of cartridge 205 through slots 208 in the distal end of cartridge 205 and engage matching depressions 210 on the face of anvil 103. Continued pulling on the wires attached to the extendable rods 207 of the anvil 103 causes more and more of the length of the legs of the staples to exit through slots 208 and layers of tissue until the legs of the staples start to curl in depressions 210. The process continues until the staples completely exit the cartridge 205, the legs are completely curled and the process of stapling the layers of tissue together is completed.

A tissue gripping device comprising at least one tissue gripper 204 is configured to be reversibly extended from base 202. In the embodiment shown in FIGS. 2A and 2B, gripper 204 is made of stiff wire, with its distal end bent into a corkscrew shape. Other embodiments of gripping means can comprise, for example, forceps or any similar grasping tool known in the art. The proximal end of gripper 204 is connected to a wire 216, which is connected to a mechanism in the handle of the endoscope or in the dedicated control box, which controls the longitudinal and the rotational motion of gripper 204. In embodiments in which base 202 is an integral part of the endoscope, wire 216 passes through channels in the insertion tube of the endoscope and in embodiments in which base 202 is added on to an endoscope, wire 216 passes through tube 218 to the proximal end of endoscope 100.

In the opened configuration shown in FIG. 2A and FIG. 2B, an operating space 206, shown as the gap between two dashed lines, exists in the space between rods 207 and between cartridge 205 and anvil 103. It is in operating space 206 that medical procedures such as hole closure or polyp removal can take place. When wire 216 is extended distally and rotated, gripper 204 is advanced in a spiral motion in order to penetrate and grip tissue (not shown) located in front of the distal face of endoscope 100. Once a firm grip on the tissue has been achieved, gripper 204 is retracted, which pulls the tissue upwards and backwards into the operating space 206.

Backward facing camera 209 allows continuous observation of procedures carried out in operating space 206, not only from a point of view not attainable by forward looking camera 101, but also when the field of view of forward facing camera 101 is completely blocked by tissue located in the operating space 206, for example when tissue surrounding a hole is pulled up by gripper 204 in order to staple the ends together and close the hole.

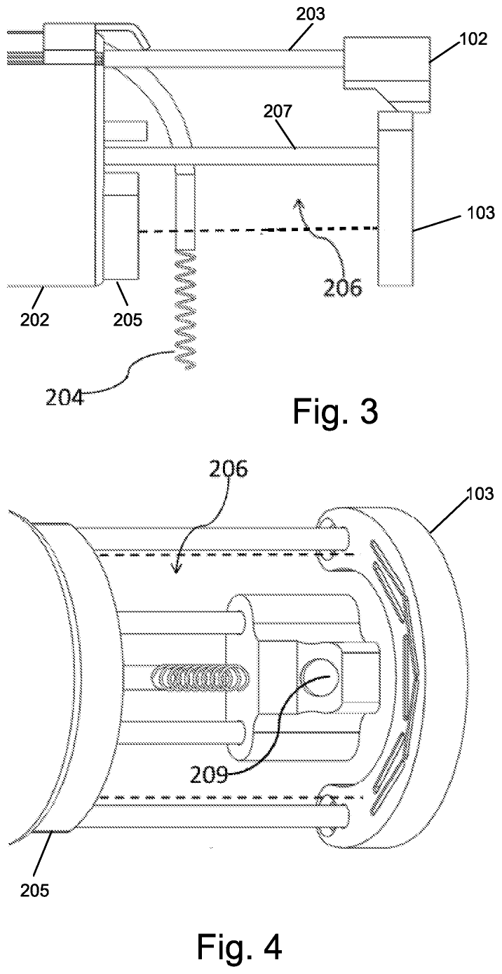

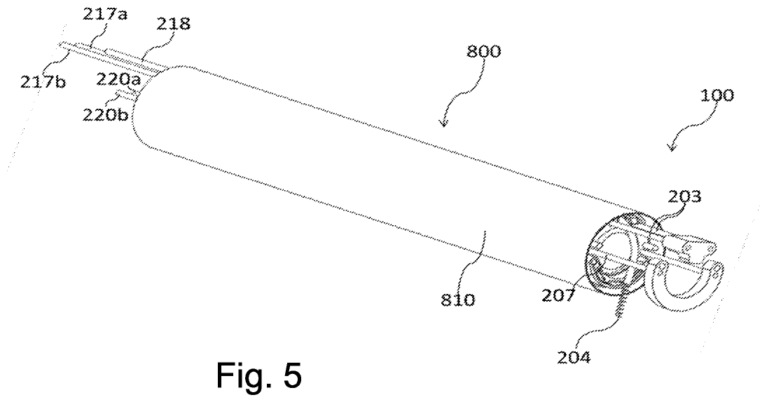

FIG. 3 and FIG. 4 respectively show a side and bottom view of endoscope 100 in its opened configuration. In FIG. 3 gripper 204 is shown extended through operating space 206. FIG. 4 shows a clear line of sight between backward facing camera 209 and operating space 206. It is noted that, although only one gripper is illustrated herein, embodiments of the ECD with more than one gripper can be provided to grab and draw tissue into operating space 206.

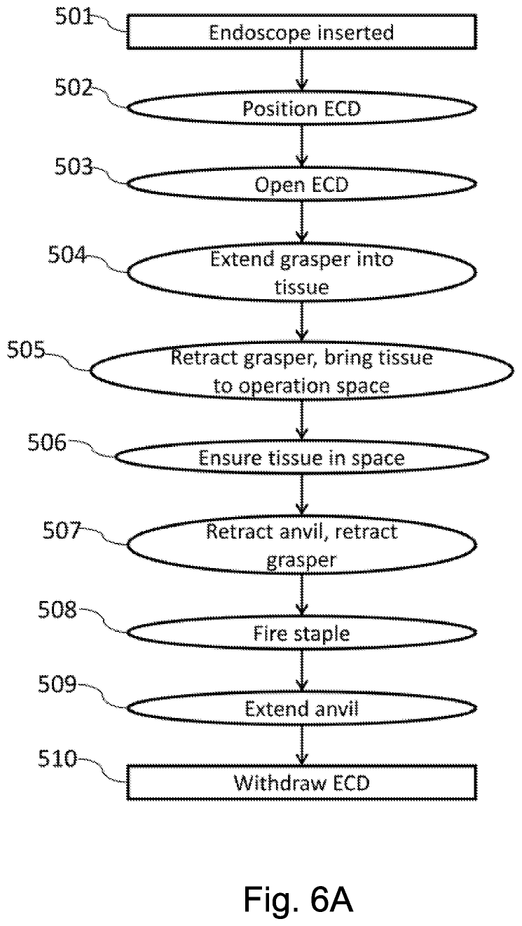

FIG. 5 schematically shows an add-on embodiment of endoscope 100. Tubes 217a, 217b, 218, 220a and 220b are shown at the proximal end of sheath 810. Wire 216 (not shown in FIG. 5), that is used to control gripper 204, passes through tube 218. Similarly, wires pass through tubes 220a and 220b that control the longitudinal motion of rods 207. Wires which control the longitudinal motion of rods 203 pass through tubes 217a and 217b.

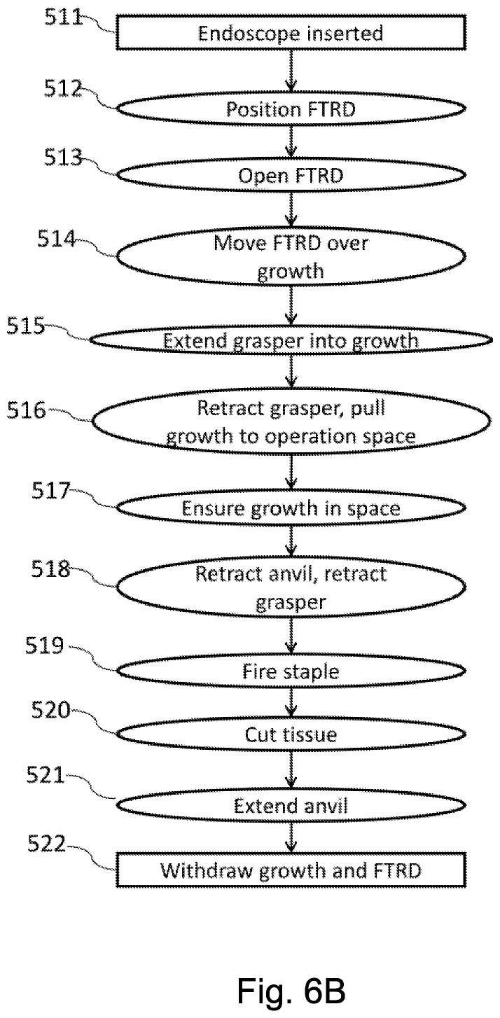

FIG. 6A is a flowchart describing an endoscopic closure process using the embodiment of an endoscopic closure device (ECD) described herein above. In the first step 501, endoscope 100 is inserted into the body of a patient and front facing camera 101 is used to guide the insertion tube to the location of a hole in the tissue that is to be closed and, in step 502, to position the distal tip above and parallel to the hole which is to be closed. In step 503, the operator actuates wires which cause anvil 103 and camera mount 102 to move distally, thereby creating operating space 206. In step 504 the operator actuates wire 216 which cause the gripper 204 to extend through the operating space 206 and to rotate in order to grip the tissue of interest. Once gripper 204 firmly grasps the tissue around the hole, in step 505 gripper 204 and attached tissue are pulled upwards and proximally through the operating space, pulling the tissue of interest through the operating space until the entirety of the hole that is to be closed is above the slots 208 in the distal end of cartridge 205. In step 506 the backward facing camera 209 is used to ensure the tissue is in the correct position relative to the staple cartridge. In step 507, the anvil is retracted towards the cartridge, pressing the tissue between the anvil and the cartridge. In the step 508, an array of staples is ejected from the cartridge through the tissue, as described herein above. In step 509, the anvil is moved distally, releasing the stapled tissue from the operating space after which the backward facing camera 209 is used to inspect and verify that the stapling procedure has been properly executed. Finally in step 510 endoscope 100 is withdrawn from the body of the patient.

The order of the steps in FIG. 6A is given just for illustration. Other orders of the steps are possible, for example steps 502 and 503 can be interchanged allowing the backward facing camera to be used to help position the ECD relative to the hole.

According to another embodiment, endoscope 100 comprises a cutting device, e.g. a blade or a heated wire, adapted to cut through tissue; thereby allowing removal of tissues, e.g. polyps, in addition to performing tissue closure. In different embodiments the cutting device is mounted on the proximal face of the anvil, on the distal face of the cartridge, or is implemented as a separate tool passed through a working channel of the endoscope.

FIG. 6B is a flowchart describing the use of endoscope 100 for performing a Full Thickness Resection (FTR) procedure to remove a growth such as a polyp or tumor. In this figure the endoscopic device 100 is known as a Full Thickness Resection Device (FTRD). In the first step 511, endoscope 100 is inserted into the body of a patient and, with the aid of front facing camera 101, guided to the location of a growth that is to be removed and in step 512 positioned above and parallel to the growth. In the step 513, the operator actuates wires which cause the anvil and camera mount to move distally thereby creating operating space 206. In step 514 the position of the endoscope is adjusted using both front facing and rear facing cameras so that the growth is below the operating space. In step 515 the operator actuates wires which cause the gripper 204 to extend through the operating space 206 and to rotate in order to grip the growth. After gripper 204 has firmly gripped the growth, in step 516, the gripper is retracted through the operating space pulling the growth through the operating space until the entire growth is above the staple cartridge and a portion of tissue below the growth is located opposite the slots 208 in the distal end of cartridge 205. At this point in the procedure, the field of view of front facing camera 101 is blocked by the growth so, in the next step 517 the backward facing camera 209 is used to ensure the growth is in the correct position. In step 518, the anvil is retracted towards the cartridge, pressing tissue below the growth between the anvil and the cartridge. In step 519, at least one staple is ejected from the cartridge through the tissue below the growth. In step 520, a cutting device is actuated by the operator and the tissue below the growth is cut above the staple line. In the next step 521, the anvil is moved distally, releasing the stapled tissue from the operating space and leaving the growth connected to the gripper. In the next and final step 522 the growth is withdrawn from the body of the patient along with endoscope 100.

An additional step can be introduced between steps 519 and 520. This additional step comprises moving the anvil distally after firing the staples to carry out a detailed inspection of the stapled tissue using backward facing camera 209 followed by moving the anvil proximally before cutting the tissue.

The order of the steps that are shown in FIG. 6B is presented just for illustration. Other orders of the steps are possible, for example steps 512 and 513 can be interchanged allowing the backward facing camera to be used to help position the endoscope relative to the growth.

In the embodiment of endoscope 100 described herein above there is a fixed distance between extendible anvil rods 207 through which growths and tissue can be pulled, in order to perform procedures such as those described in FIG. 6A and FIG. 6B. This distance is determined by the maximum diameter of the insertion shaft of endoscope 100, which in turn is determined by the minimum inner diameter of the lumen through which the insertion shaft must travel to reach the site at which the procedure is to be carried out. Therefore, in some instances, a growth or a hole with a diameter that is larger than the opening between rods 207 cannot be successfully operated upon because it cannot be pulled entirely into the operating space between the rods.

FIG. 7 (see drawing sheet 2/11) is a perspective view of anvil 103 attached to base 202 by rods 401 that are configured to provide a solution to the problem of the limited distance between them. As in the case of rods 207 described herein above, the proximal ends of rods 401 are connected to the distal ends of wires that control movement of the anvil distally and proximally from handle of the endoscope or a dedicated box. These wires pass through tubes in the interior of the insertion tube of endoscope 100 or through tubes 220a and 220b in an add-on embodiment. When the wires are pulled proximally, the rods 401 are in a first position confined inside the tubes in a straight configuration. As they are pushed distally and they exit the tubes their pre-shaped memory property causes them to bow outward until they reach a second position having the shape shown in FIG. 7 when they are totally extended in front of base 202.

After positioning ECD 100 above a tissue of interest and extending rods 401, tissue is grabbed by gripper 204 and is pulled it into operating space 206. After the tissue is grabbed and pulled into the operating space, rods 401 are retracted proximally, pulling them into tubes 220a and 220b. As the rods 401 enter the tubes, the rods are straightened and tissue encompassed by rods 401 is squeezed between them, until the tissue is finally pressed between cartridge 205 and anvil 103 for stapling, as described above.

Embodiments of the endoscopic device of the invention can be usefully employed to perform the visualization functions of robotic surgical systems.

Although embodiments of the invention have been described by way of illustration, it will be understood that the invention may be carried out with many variations, modifications, and adaptations, without exceeding the scope of the claims.

1. An endoscopic device comprising an insertion tube, a distal tip, and at least one camera located at an initial location on or in the distal tip or the insertion tube;
the endoscopic device characterized in that it comprises a mechanical arrangement that is activated from a proximal end of the endoscopic device, the mechanical arrangement configured to do at least one of the following:
(a) to move the at least one camera from the initial location to one or more different locations on, in or spaced away from the distal tip or the insertion tube; and
(b) to change a direction of view of the at least one camera.

2. The endoscopic device of claim 1 comprising a first set of cameras comprising one or more cameras that are fixedly attached to the distal tip or insertion tube of the endoscopic device and a second set of cameras comprising one or more cameras each of which is mounted on at least one rod configured to be reversibly extended outwards from the distal tip or insertion tube of the endoscopic device, wherein the first set of cameras provides a direction of view looking outwards from the interior of the endoscopic device and the second set of cameras provides a direction of view looking inwards towards the endoscopic device.

3. The endoscopic device of claim 2, wherein the at least one rod is configured to be reversibly extended in any direction with respect to the surface of the endoscope in order to provide a field of view that cannot be obtained by cameras that are attached fixedly to the endoscope.

4. The endoscopic device of claim 1, wherein at least one camera is mounted in such a way that the camera can be panned, tilted, or rotated in order to provide various angles of view without rotating or moving the endoscope.

5. The endoscopic device of claim 2, wherein the first set of cameras comprises one front facing camera fixedly attached to distal face of the distal tip and the second set of cameras comprises one backward facing camera mounted on a camera mount attached to at least one rod that can be extended from a first position abutting a base to a second position spaced apart from the base and retracted proximally from the second position back to the first position.

6. The endoscopic device of claim 5, wherein the base is one of:
a) a specially configured distal tip, which is an integral part of a dedicated endoscope, wherein the distal tip comprises at least one bore through which the at least one rod can be slid distally and proximally; and
b) a collar which slips over and is attached to the distal tip of an endoscope, wherein the collar comprises at least one bore through which the at least one rod can be slid distally and proximally.

7. The endoscopic device of claim 5, comprising a stapling device comprised of an anvil and a staple cartridge, wherein the anvil is mounted on two rods which can be extended distally from the first position to the second position to obtain an opened configuration or can be retracted proximally from the second position to the first position to obtain a closed configuration; and wherein the staple cartridge comprises a distal section configured to slide proximally into a proximal section of the staple cartridge.

8. The endoscopic device of claim 7, comprising at least one tissue gripping device comprising at least one tissue gripper configured to be reversibly extended from the base.

9. The endoscopic device of claim 8, comprising at least one cutting device configured to cut through tissue.

10. The endoscopic device of claim 9, wherein the anvil is attached to two rods that have a straight configuration when in the closed configuration and a bowed configuration when in the open configuration.

11. A method of using the endoscopic device of claim 8 for performing an endoscopic closure procedure, said method comprising the steps:
a) inserting an endoscopic device into the body of a patient and using the front facing camera to guide the insertion tube to a location of a hole, which is to be closed, in tissue of a patient;
b) using the front facing camera to position the distal tip of the endoscopic device above and parallel to the hole, which is to be closed;
c) actuating wires which cause the anvil of the stapling device and the camera mount to move distally;
d) actuating the at least one tissue gripper to extend and firmly grasp tissue around the hole;
e) pulling the tissue gripper and attached tissue upwards and proximally until all tissue surrounding the hole that is to be closed is above slots through which staples in stored in the proximal section of the staple cartridge are ejected from the distal section of the staple cartridge;
f) using the backward facing camera to ensure the tissue is correctly positioned relative to the staple cartridge;
g) retracting the anvil towards the staple cartridge thereby pressing the tissue surrounding the hole between the anvil and the staple cartridge;
h) continuing to retract the anvil towards the staple cartridge, thereby ejecting an array of staples from the staple cartridge through the tissue;
i) moving the anvil distally thereby releasing the stapled tissue and using the backward facing camera to inspect and verify that a stapling procedure has been properly executed; and
j) withdrawing the endoscope from the body of the patient.

12. A method of using the endoscopic device of claim 9 for performing a Full Thickness Resection (FTR) procedure to remove a growth, the method comprising the steps:
a) inserting the endoscope into the body of a patient and using the front facing camera to guide the endoscope to a location of a growth that is to be removed;
b) using the front facing camera to position the endoscope above and parallel to the growth;
c) actuating wires which cause the anvil and the camera mount to move distally;
d) adjusting the position of the endoscope using both the front facing camera and a rear facing camera so that the growth is correctly positioned below and in front of the distal tip of the endoscope;
e) actuating the tissue gripper to firmly grip the growth;
f) retracting the gripper, thereby pulling the entire growth above the staple cartridge until a portion of tissue surrounding the growth is located opposite slots through which staples in the proximal section of the staple cartridge are ejected from the distal section of the staple cartridge;
g) using the backward facing camera to ensure that the growth is in the correct position;
h) retracting the anvil towards the staple cartridge thereby pressing tissue surrounding the growth between the anvil and the staple cartridge;
i) continuing to retract the anvil towards the staple cartridge, thereby ejecting at least one staple from the staple cartridge through the tissue surrounding the growth;
j) actuating the cutting device thereby cutting the tissue surrounding the growth below a staple line;
k) moving the anvil distally thereby releasing the stapled tissue while leaving the growth connected to the gripper; and
l) withdrawing the growth together with the endoscope from the body of the patient.

13. The method of claim 12 comprising an additional step introduced between steps i and j, wherein the additional step comprises moving the anvil distally after firing the staples to carry out a detailed inspection of the stapled tissue using the backward facing camera followed by moving the anvil proximally before cutting the tissue.

14. The endoscopic device of claim 1 wherein the mechanical arrangement is a ball joint located on a distal face of the distal tip, the ball joint comprising a ball on which the at least one camera and illumination sources are mounted; wherein the ball is configured to be rotated by an operator of the endoscopic device by activating cables or an electric motor and gear system, thereby changing the direction of view of the at least one camera.

15. The endoscopic device of claim 1 wherein the mechanical arrangement is a rotatable collar on which is mounted the at least one camera and illumination sources; wherein the collar is configured to be rotated by an operator in a clockwise or counterclockwise direction around a longitudinal symmetry axis of the insertion tube by rotating a cable connected to the collar or by activating an electric motor and gear system, thereby changing the direction of view of the at least one camera.

16. The endoscopic device of claim 15 comprising at least one additional camera on the distal tip; wherein the at least one additional camera has one of:
a) a fixed front facing direction of view; and
b) a mechanical arrangement configured to change its direction of view.

17. A robotic surgical system comprising at least one endoscopic device of claim 1.

18. A method of using the endoscopic device of claim 10 for performing a Full Thickness Resection (FTR) procedure to remove a growth, the method comprising the steps:
a) inserting the endoscope into the body of a patient and using the front facing camera to guide the endoscope to a location of a growth that is to be removed;
b) using the front facing camera to position the endoscope above and parallel to the growth;
c) actuating wires which cause the anvil and the camera mount to move distally;
d) adjusting the position of the endoscope using both the front facing camera and a rear facing camera so that the growth is correctly positioned below and in front of the distal tip of the endoscope;
e) actuating the tissue gripper to firmly grip the growth;
f) retracting the gripper, thereby pulling the entire growth above the staple cartridge until a portion of tissue surrounding the growth is located opposite slots through which staples in the proximal section of the staple cartridge are ejected from the distal section of the staple cartridge;
g) using the backward facing camera to ensure that the growth is in the correct position;
h) retracting the anvil towards the staple cartridge thereby pressing tissue surrounding the growth between the anvil and the staple cartridge;
i) continuing to retract the anvil towards the staple cartridge, thereby ejecting at least one staple from the staple cartridge through the tissue surrounding the growth;
j) actuating the cutting device thereby cutting the tissue surrounding the growth below a staple line;
k) moving the anvil distally thereby releasing the stapled tissue while leaving the growth connected to the gripper; and
l) withdrawing the growth together with the endoscope from the body of the patient.

19. The method of claim 18 comprising an additional step introduced between steps i and j, wherein the additional step comprises moving the anvil distally after firing the staples to carry out a detailed inspection of the stapled tissue using the backward facing camera followed by moving the anvil proximally before cutting the tissue.
